(19) United States
(12) Patent Application Publication
(10) Pub. No.: US 2026/0165796 A1
(43) Pub. Date: Jun. 18, 2026

(54) SYSTEMS AND METHODS FOR ROBOTIC ENDOLUMINAL SUTURING INSTRUMENT

(71) Applicant: Noah Medical Corporation, San Carlos, CA (US)

(72) Inventors: Matthew Robert Penny, Holly Springs, NC (US); Neil Markwardt, Redwood City, CA (US)

(21) Appl. No.: 19/532,752

(22) Filed: Feb. 6, 2026

Related U.S. Application Data
(63) Continuation of application No. PCT/US2024/043223, filed on Aug. 21, 2024.
(60) Provisional application No. 63/578,829, filed on Aug. 25, 2023.

(51) Int. Cl. A61B 34/30; A61B 17/04

(52) U.S. Cl. CPC A61B 34/30; A61B 17/0469; A61B 2034/301; A61B 2034/305

(57) ABSTRACT

A suturing instrument is provided. The suturing instrument comprises: an elongate member comprising an articulatable bending section and a shaft; and a needle end effector comprising a distal portion located at a distal end of the bending section, and a flexible construction of the needle is extended along the entire length of the bending section.

### REFERENCE

This application is a continuation of International Application No. PCT/US2024/043223, filed on Aug. 21, 2024, which claims priority to U.S. Provisional Patent Application No. 63/578,829, filed on Aug. 25, 2023, which is entirely incorporated herein by reference.

### BACKGROUND

Endoscopy procedures use an endoscope to examine the interior of a hollow organ or cavity of the body. Unlike many other medical imaging techniques, endoscopes are inserted into the organ directly via the mouth or other naturally occurring orifices. Flexible endoscopes that can deliver instinctive steering and control are useful in diagnosing and treating diseases that are accessible through any natural orifice in the body. Depending on the clinical indication, the endoscope may be designated as colonoscope, gastroscope, bronchoscope, ureteroscope, ENT scope, and various others. For example, a flexible colonoscope may be intubated to transverse colon for diagnosis and/or surgical treatment.

An endoscope device may have a working channel allowing tools such as graspers, cutters or suturing instruments to pass through. In another example, a suturing device can be coupled to the distal end of an endoscope, which enables suturing in the gastroesophageal tract of a patient. The flexible endoscope may have a bending section and a rigid tip or end effector attached to the bending section. In order to be able to be maneuvered through bodily lumens, the dimension (length) of the digital tip portion of a flexible endoscope is desired to be as small as possible so that it can pass through tortious pathways. However, current suturing devices are designed for manual endoscope devices such as laparoscopy which typically has a length or rigid structure greater than a desired dimension for an end effector. In laparoscopic surgery, suturing is one of the most difficult surgical tasks to perform and there have been several innovations to make it easier for clinicians. In endoscopic surgery, surgical tasks, including suturing, are substantially more challenging due to the limited space, tortuous anatomy and the compliance of the tools. Therefore, it is desirable to provide suturing devices suitable for robotic endoscopic platforms or used endoluminally.

### SUMMARY

Recognized herein is a need for suturing devices suitable for robotic endoscopic platforms or used endoluminally with reduced size and improved efficiency in workflow. The present disclosure provides an improved suturing instrument that can streamline a suturing workflow (e.g., close wounds) in a bodily lumen (e.g., the colon). The suturing instrument herein may have a compact design with reduced diameter (e.g., no greater than 3 mm, 4 mm, 5 mm, 6 mm, 7 mm, etc.) and reduced end effector length compared to traditional suturing instrument. The end effector length may be reduced by providing a flexible construction in the suturing device allowing it to be placed through a flexible endoscope (e.g., colonoscope) and can be robotically controlled in tortuous anatomy. Unlike traditional suturing instrument where a needle driver is attached to a bending section at the distal end acting as an end effector, the flexible construction of the suturing instrument herein may beneficially allow at least a portion of a needle driver to be placed within a bending section of the instrument or colocalized with a bending section such that the effective length of the end effector is reduced.

In some embodiments, the suturing instrument or suturing device may comprise a needle and suture. Unlike traditional device that typically has a free needle and needle driver which requires introducing suture and needle separately, by including both needle and suturing in the suturing device it beneficially simplifies the streamline for robotic suturing operations. Additionally, the needle may be retained completely within the suturing device thereby reducing the risk of damaging the needle, colonoscope or tissue during the needle introduction and extraction from the surgical field.

In an aspect of the present disclosure, a suturing instrument is provided. The suturing instrument comprises: an elongate member comprising an articulatable bending section and a shaft; and an end effector comprising a rigid distal portion located at a distal end of the bending section, and a flexible shaft of a needle extended along an entire length of the bending section.

In some embodiments, the rigid distal portion comprises a ferrule retention mechanism configured to strip a ferrule from the needle. In some cases, the ferrule retention mechanism comprises a toggle to actuate a lever into a ferrule release position and a ferrule retention position. For instances, the ferrule retention mechanism is independently controlled relative to a needle driving mechanism.

In some embodiments, a needle driving mechanism is located approximately at a distal end of the shaft or at an interface between a proximal end of the articulatable bending section and the distal end of the shaft. In some cases, the needle driving mechanism is connected to the flexible shaft of the needle. For instances, the flexible shaft of the needle is located at an offset from a neutral axis of the bending section. In some cases, the flexible shaft of the needle adapts to a path length change when the bending section is articulated. In some examples, a tension in a cable for retracting the needle is maintained by adapting to the path length change.

In some embodiments, the suturing instrument further comprises a handle portion releasably attached to an instrument driving mechanism. In some embodiments, the instrument driving mechanism is supported by a robotic arm. In some cases, the robotic arm is configured to further support another instrument driving mechanism that is releasably attached to a handle portion of an endoscope. In some cases, the suturing instrument is inserted through a working channel of the endoscope.

In another aspect of the present disclosure, a suturing instrument is provided. The suturing instrument comprises: a flexible elongate member comprising an articulatable bending section connected to a distal end of a shaft; and an end effector comprising a rigid distal portion located at a distal end of the bending section, and a needle driving mechanism for driving a needle, wherein the needle driving mechanism is located at the distal end of the shaft or at an interface between a proximal end of the articulatable bending section and the distal end of the shaft.

In some embodiments, the rigid distal portion comprises a ferrule retention mechanism configured to strip a ferrule from the needle. In some cases, the ferrule retention mechanism comprises a toggle to actuate a lever into a ferrule release position and a ferrule retention position. In some instances, the ferrule retention mechanism is independently controlled relative to the needle driving mechanism.

In some embodiments, the needle driving mechanism is connected to a flexible shaft of the needle. In some cases, the flexible shaft of the needle is extended along an entire length of the bending section. In some instances, the flexible shaft of the needle is at an offset from a neutral axis of the bending section. For example, the flexible shaft of the needle adapts to a path length change when the bending section is articulated. In some cases, a tension in a cable for retracting the needle is maintained by adapting to the path length change.

In some embodiments, the suturing instrument further comprises a handle portion releasably attached to an instrument driving mechanism. In some embodiments, the instrument driving mechanism is supported by a robotic arm. In some cases, the robotic arm is configured to further support another instrument driving mechanism that is releasably attached to a handle portion of an endoscope. In some cases, the suturing instrument is inserted through a working channel of the endoscope

It should be noted that the provided suturing device, end effector, endoscope components and various components of the device can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others. The devices and systems can be used in any subject that may or may not involve human body, animal, or tissue.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically shows an example of a suturing instrument, in accordance with some embodiments of the present disclosure.

FIG. 2 shows an example of an end effector located at a distal portion of a flexible suturing instrument, in accordance with some embodiments of the present disclosure.

FIG. 3 and FIGS. 4A-4B show examples of a substantially rigid portion of the end effector.

FIG. 5 shows a top view and a bottom view of an active ferrule retention mechanism.

FIG. 6 shows examples of a ferrule retention lever is positioned at a “ferrule retention” location and a ferrule release location.

FIG. 7 shows an example of a needle having a flexible construction, in accordance with some embodiments of the present disclosure.

FIGS. 8A-8B show examples of a needle at least partially located within a bending section.

FIG. 9 and FIG. 10 show an example of a needle drive mechanism.

FIG. 11 shows different engagement features of needles and ferrules.

FIGS. 12A-12B shows an exemplary sequence of operations for throwing a stitch.

FIG. 13 shows an example of a robotic support system comprising instrument driving mechanism (IDM) for a suturing instrument and IDM for an endoscope.

FIG. 14 shows examples of instrument driving mechanism (IDM).

FIG. 15 illustrates an example of a flexible endoscope, in accordance with some embodiments of the present disclosure.

FIG. 16 illustrates an example of a distal tip of a flexible endoscope, in accordance with some embodiments of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides an improved suturing instrument that can streamline a suturing workflow (e.g., close wounds) in a bodily lumen (e.g., the colon). The suturing instrument herein may have a compact design with reduced diameter (e.g., no greater than 3 mm, 4 mm, 5 mm, 6 mm, 7 mm, etc.) and reduced end effector length. The end effector length may be reduced by providing a flexible construction in the suturing device allowing it to be placed through a flexible endoscope (e.g., colonoscope) and can be robotically controlled in tortuous anatomy. Unlike traditional suturing instrument where a needle driver is attached to a bending section at the distal end acting as an end effector, the flexible construction of the suturing instrument herein may beneficially allow at least a portion of a needle driver to be placed within a bending section of the instrument or colocalized with a bending section such that the effective length of the end effector is reduced.

In some embodiments, the suturing instrument or suturing device may comprise a needle and suture. Unlike traditional device that typically has a free needle and needle driver which requires introducing suture and needle separately, by including both needle and suturing in the suturing device it beneficially simplifies the streamline for robotic suturing operations. Additionally, the needle may be retained completely within the suturing device thereby reducing the risk of damaging the needle, colonoscope or tissue during the needle introduction and extraction from the surgical field.

Current needle devices may achieve a stitch cycle including traversing a needle, picking up a ferrule, the ferrule being returned to its ferrule compartment and the ferrule being stripped. Existing needle devices may rotate the needle into various angles in order to engage and disengage the needle tip with the ferrule. For example, by orienting a faceted edge of a needle, the faceted edge may engage and release from a ferrule latch. The suturing device herein may provide a stitch cycle without rotating the needle. In some embodiments, the suturing device may comprise an “active” needle retention mechanism that can be toggled between one of two states: (1) ferrule retained and (2) ferrule released. The active needle retention mechanism allows the ferrule to be released from the distal pocket at any time without having to be coupled to a motion of the needle.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of tissues and organs, for example. It is to be understood that any one or more of the structures and steps as described herein can be combined with any one or more additional structures and steps of the methods and apparatus as described herein, the drawings and supporting text provide descriptions in accordance with embodiments.

While exemplary embodiments will be primarily directed at a suturing device or system for colonoscope or gastroscope, one of skill in the art will appreciate that this is not intended to be limiting, and the devices described herein may be used for other therapeutic or diagnostic procedures and in various anatomical regions of a patient's body. The provided suturing device or system can be utilized in urology, gynecology, rhinology, otology, laryngoscopy, gastroenterology with the endoscopes, combined devices including endoscope and instruments, endoscopes with localization functions, one of skill in the art will appreciate that this is not intended to be limiting, and the devices described herein may be used for other therapeutic or diagnostic procedures and in other anatomical regions of a patient's body, such as such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat, and various others, in the forms of: BronchoScope, NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, Orthopedic Endoscopes, and others, in combination with various tools or instruments.

The systems and apparatuses herein can be combined in one or more of many ways to provide improved diagnosis and therapy to a patient. Systems and apparatuses provided herein can be combined with existing methods and apparatus to provide improved diagnosis, surgery operations of various tissues and organs, for example. It is to be understood that any one or more of the structures and steps as described herein can be combined with any one or more additional structures and steps of the methods and apparatus as described herein, the drawings and supporting text provide descriptions in accordance with embodiments.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms distal and proximal may generally refer to locations referenced from the apparatus, and can be opposite of anatomical references. For example, a distal location of a primary shaft or catheter may correspond to a proximal location of an elongate member of the patient, and a proximal location of the primary sheath or catheter may correspond to a distal location of the elongate member of the patient.

### Suturing Instrument

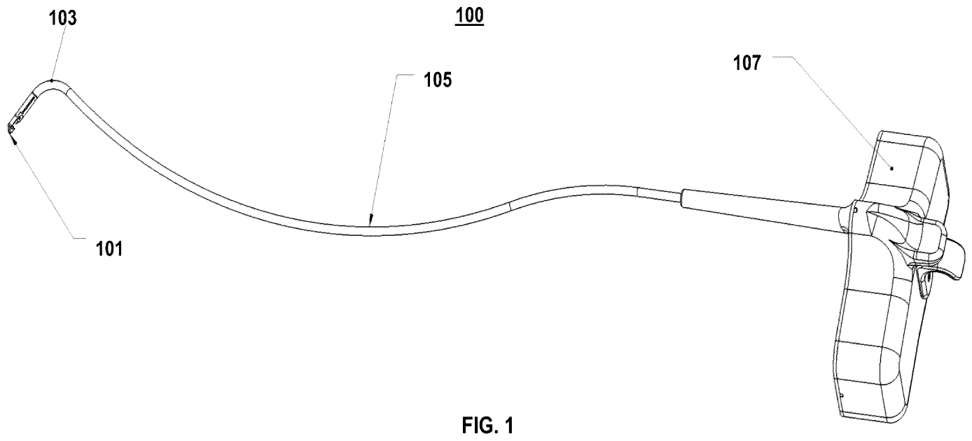

FIG. 1 schematically shows an example of a suturing instrument 100. The suturing instrument 100 may comprise an end effector 101 located at a distal end of an elongate member. The elongate member may be an articulatable, flexible member comprising a bending section 103, a shaft 105 and a proximal end 107. The proximal end 107 may be a handle that is releasably attached to a robotic support. In some cases, the proximal end 107 may comprise driving components (e.g., pulley) that are releasably coupled to an instrument driving mechanism (IDM) to drive an operation of the end effector (e.g., needle operation, ferrule retention mechanism operation, etc.) and/or the motion (e.g., articulation) of the bending section 103.

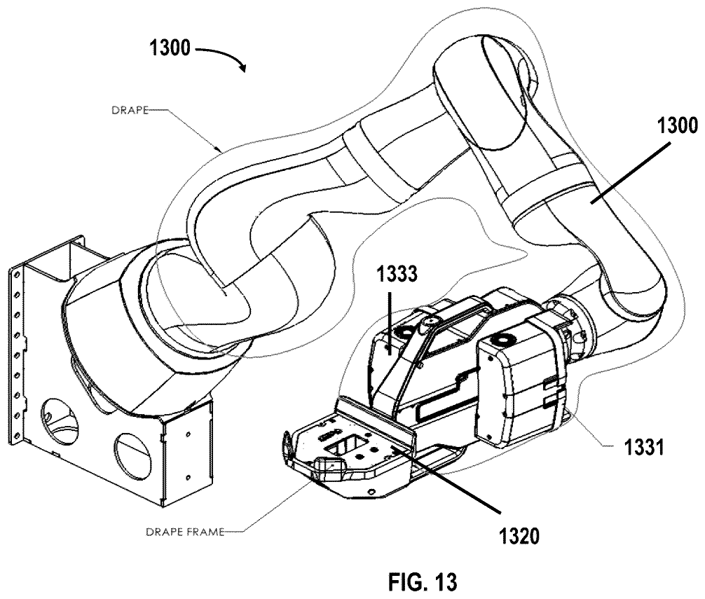

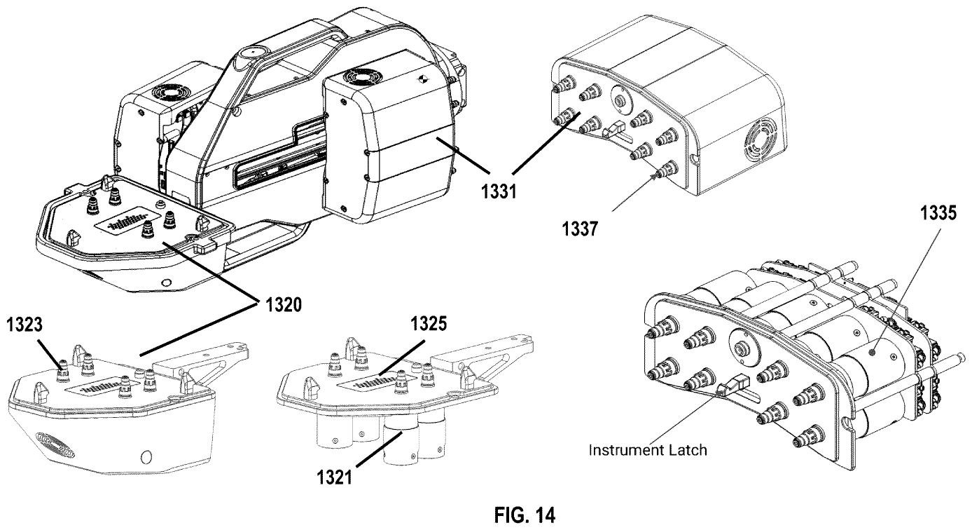

In some cases, the proximal end 107 may comprise a mechanical interface to allow the suturing instrument to be releasably coupled to an instrument driving mechanism attached to a robotic support or a hand-held controller. The instrument driving mechanism (IDM) can be the same as the IDM 1331, 1333 as illustrated in FIG. 13 and FIG. 14. The IDM may comprise a set of motors 1335 that are actuated to rotationally drive a set of pull wires of the elongate member to control an articulation of the bending section, as well as controlling an operation of the end effector such as the needle displacement motion and ferrule retention mechanism operation (e.g., rotation of the ferrule retention door). The proximal end 107 may be mounted onto the instrument drive mechanism 1331 or 1333 so that its pulley/capstans assemblies are driven by the set of motors 1335. The number of pulleys may vary based on the pull wire configurations. In some cases, one, two, three, four, or more pull wires may be utilized for articulating the flexible suturing instrument (e.g., bending section articulation in two or more degree of freedoms) and for driving the motion of the needle end effector. In some cases, one pull wire may be coupled to and driven by a pulley. In some cases, more than one wires may be coupled to a driven pulley. For example, two or more wires may be coupled to the same driven pulley antagonistically to drive the needle end effector motion such that rotation of the pulley provides tension to one wire(s) while slacking the other(s).

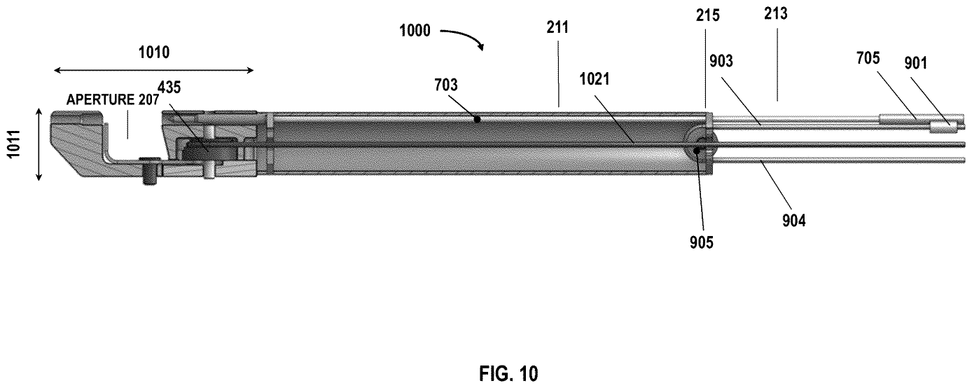

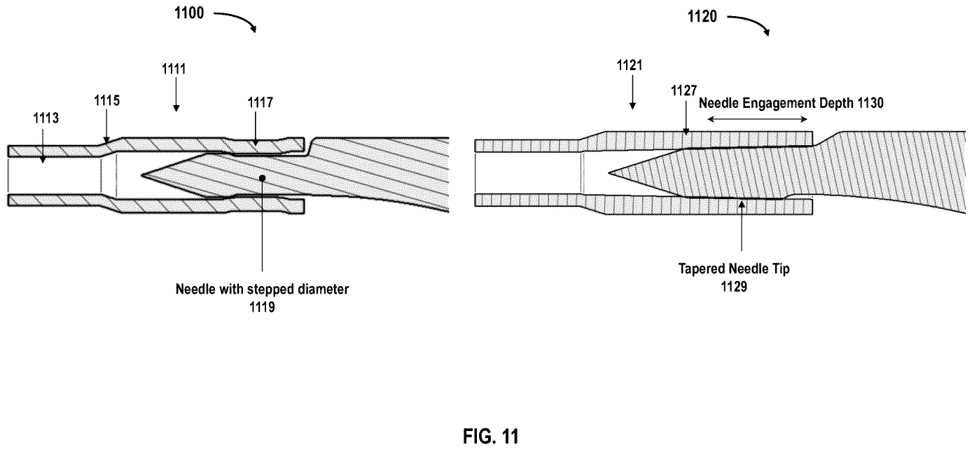

The bending section 103 may be articulated in one or more degrees of freedom. The articulation of the bending section 103 may be controlled by applying force to the distal tip portion of the elongate member via the one or multiple pull wires. A distal end of the one or more pull wires may be attached to the distal end of the suturing instrument 100. In the case of multiple pull wires, pulling one wire at a time may change the orientation of the end effector 101 to pitch up, down, left, right or any direction needed. In some cases, the pull wires may be anchored at the distal tip portion of the suturing instrument 100, running through the bending section, and entering the proximal end they are coupled to a driving component (e.g., pulley). This pulley may interact with an output shaft (e.g., output shaft 1337 in FIG. 14) from the robotic system. In some cases, one or more of the pull wires may be utilized as the needle drive mechanism (e.g., needle retraction cable and needle insertion cable in FIG. 10 and FIG. 11) to drive a translational or displacement movement of the needle (e.g., drive forward and backward motion of the needle in the end effector).

In some embodiments, the proximal end or portion of one or more pull wires may be operatively coupled to various mechanisms (e.g., gears, pulleys, capstans, etc.) in the proximal end. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the suturing instrument 100, such that operation of the pull wires by the control unit may apply force or tension to the distal portion which may steer or articulate (e.g., up, down, pitch, yaw, or any direction in-between) at least the distal portion (e.g., bending section) of the suturing instrument. The pull wires may be made of any suitable material such as stainless steel (e.g., SS316), metals, alloys, polymers, nylons or biocompatible material. In some embodiments, different pull wires may be made of different materials for varying the load bearing capabilities of the pull wires.

The shaft 105 may connect the bending section 103 to the proximal handle 107. The shaft 105 may be flexible and a passive section. For example, the active bending section 103 may be designed to allow for bending in two or more degrees of freedom (e.g., articulation). A greater bending degree such as 180 and 270 degrees (or other articulation parameters for clinical indications) can be achieved by the unique structure of the active bending section 103. In some cases, the active bending section 103 and the passive section or shaft 105 may be fabricated separately as a modular component and assembled to the elongate member. In some cases, the cut patterns of the active bending and shaft may be different such that at least the minimum bend radius of the two sections may be different. In some cases, a variable minimum bend radius along the axial axis of the elongate member may be provided such that an active bending section 103 or the passive section 105 may comprise two or more different minimum bend radii. In some cases, the active bending section 103 and the shaft 105 of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

The end effector 101 may comprise a needle drive mechanism. The needle drive mechanism may be located at the distal portion of the suturing instrument 100. The needle drive mechanism may be partially located within a bending section of the suturing instrument. As described above, unlike traditional suturing instrument where the end effector is attached to a bending section at the distal end, the needle drive mechanism of the end effector 101 may comprise a flexible construction beneficially allowing at least a portion of the needle drive mechanism or the end effector to be placed within a bending section of the instrument or colocalized with a bending section such that the effective length or a length of the rigid portion of the end effector is reduced. Details about the flexible construction and the needle drive mechanism are described later herein.

The end effector 101 may have two (e.g., roll and translation), three (e.g., roll and articulation), four (e.g., roll, articulation and translation) or more degrees of freedom. For example, the end effector may have a roll movement (e.g., rotatable about the longitudinal axis of the elongate member), articulatable about two axes (e.g., via the articulation of the bending section). The end effector may also have translational movement (e.g., insertion and retraction of the needle). In some cases, the roll movement of the end effector may be achieved via a wrist at the distal portion of the suturing instrument such that the end effector 101 may have a roll movement relative to the elongate member. Alternatively, the end effector 101 may not have a roll movement relative to the bending section 103. In some cases, the roll movement of the end effector may be achieved via the roll movement of the elongate member, a wrist located at the distal end of the bending section 103 or a combination of both.

The present disclosure provides a needle end effector with reduced size (e.g., effective length of end effector or the rigid length) and improved operation control that can be used with a robotic endoscope or colonoscope to perform operations related to suturing such as needle introduction and retrieval as well as needle throwing during upper and lower gastrointestinal (GI) tract endoscopy, gastric endoscopy, small bowel endoscopy or other procedures. The reduced size of the end effector may beneficially ease insertion, manipulation, and retraction of an endoscope during colonoscopy. In particular, the devices and methods of the present disclosure provides an improved suturing instrument with an active ferrule retention mechanism allowing for improved adaptation to needles and ferrule tips of various dimensions or shapes. Additionally, the overall reduced dimension (e.g., diameter) of the suturing instrument may allow it to be used in conjunction with a robotic endoscope device (e.g., passing through a working channel of the endoscope device) for robotic endoluminal surgical platforms.

Current needle devices may achieve a stitch cycle including traversing a needle, picking up a ferrule, the ferrule being returned to its ferrule compartment and the ferrule being stripped. Existing needle devices may rotate the needle into various angles in order to engage and disengage the needle tip with the ferrule. For example, by orienting a faceted edge of a needle, the faceted edge may engage and release from a ferrule latch. The needle end effector as described herein may allow for engaging and disengaging the needle and ferrule without rotation motion which beneficially simplifies the workflow of operation which is suitable for automated robotic control.

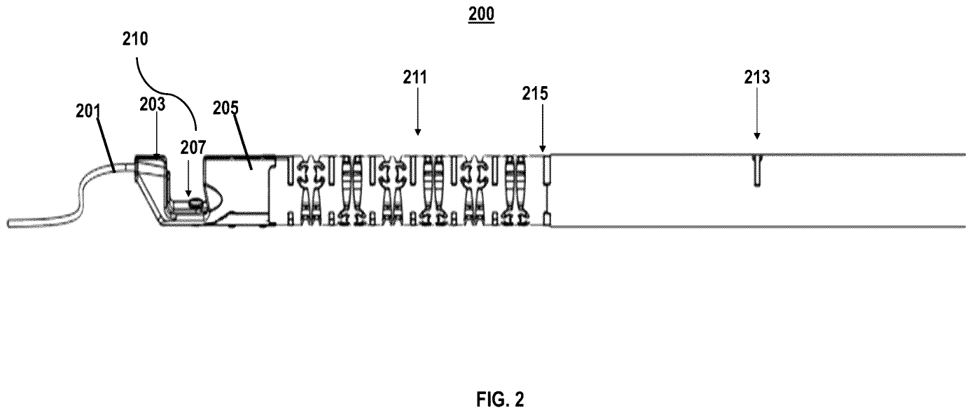

The needle instrument of the present disclosure may provide an end effector comprising active ferrule retention mechanism for engaging and disengaging the needle and ferrule. FIG. 2 shows an example of an end effector 210 located at a distal portion of a flexible suturing instrument 200. The suturing instrument 200 can be same as the suturing instrument described in FIG. 1. At least a portion of the end effector 210 is located at a distal end of the bending section 211 and a portion of the end effector is colocalized with the bending section. The bending section 211 may be connected to a shaft 213 which can be the same as the bending section and shaft as described in FIG. 1. For example, the long, flexible instrument shaft 213 may be connected to the bending section 211 at the distal end for controlling the position and orientation of the end effector 210. The proximal end of the shaft 213 is coupled to a handle for controlling the suturing instrument 200. And the handle may be releasably attached to a robotic surgical system or a controller device for manual use.

In some cases, the portion of the end effector that is colocalized with the bending section 211 may include a needle drive mechanism and/or a portion of the needle. In some cases, the needle may comprise a flexible shaft that runs along the bending section and is driven by a needle drive mechanism that is located substantially at the interface 215 between the bending section and the shaft. As described above, by arranging a portion of the end effector (e.g., needle drive mechanism) to the bending section, a length of the end effector or a length of a rigid portion is substantially reduced (e.g., reduced by at least 10%, 20%, 30%, 40%, 50%, etc.) compared to an end effector having the needle drive mechanism located within the rigid portion. A shorter end effector distal to the bending section of the instrument beneficially allows the instrument end effector to achieve more clinically relevant angles on tissue and improves the accuracy of needle tip placement. Details about the needle flexible construction and the needle drive mechanism are described later herein.

The end effector 210 may comprise a proximal portion 205 which houses an active ferrule retention mechanism and a translating needle, a distal portion 203 which provides a ferrule stowage location and an aperture 207 between the proximal portion and the distal portion. A rigid portion of the end effector is located at the distal end of the bending section. A suture ferrule may be attached to suture 201 at a distal end and the suture ferrule may be secured in a pocket at the distal portion 203 by the ferrule retention mechanism.

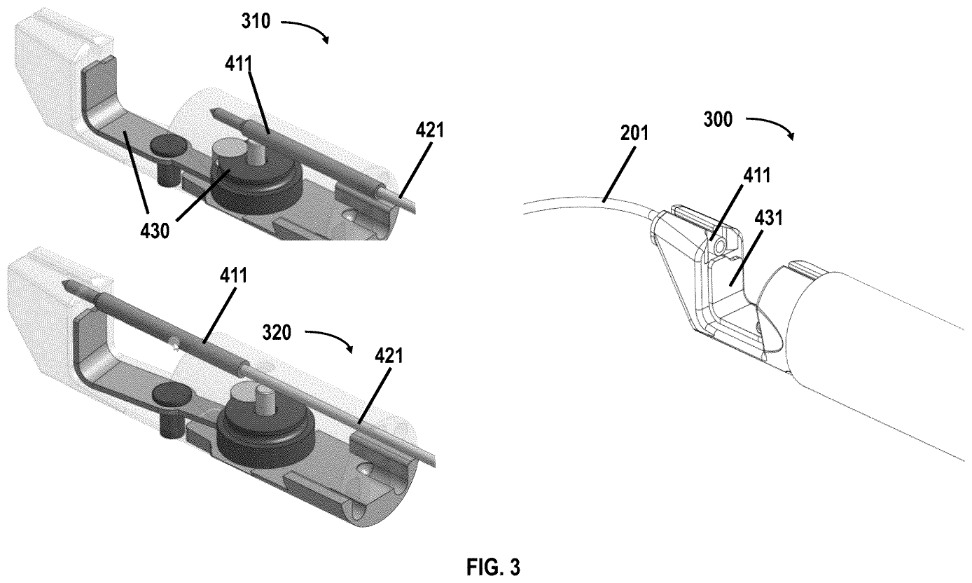

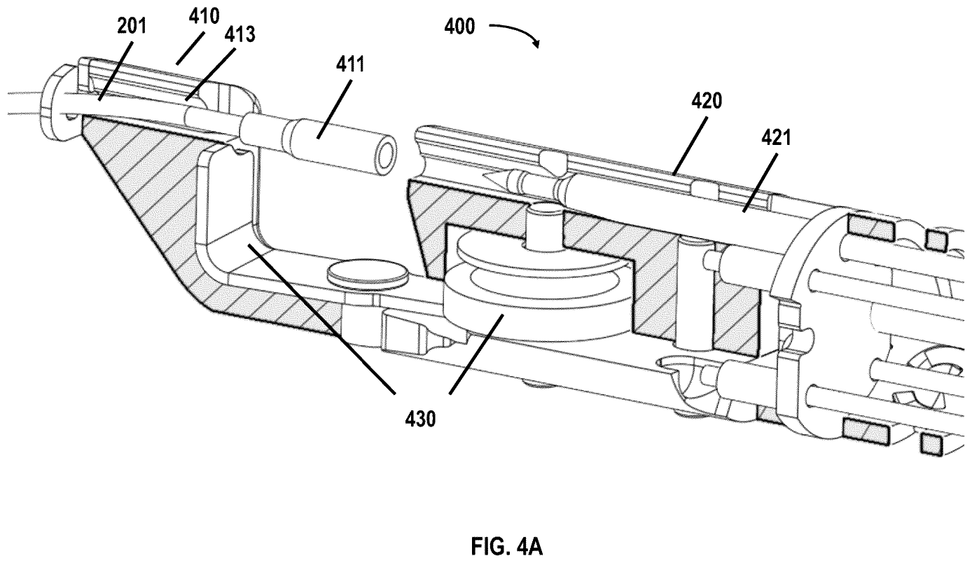

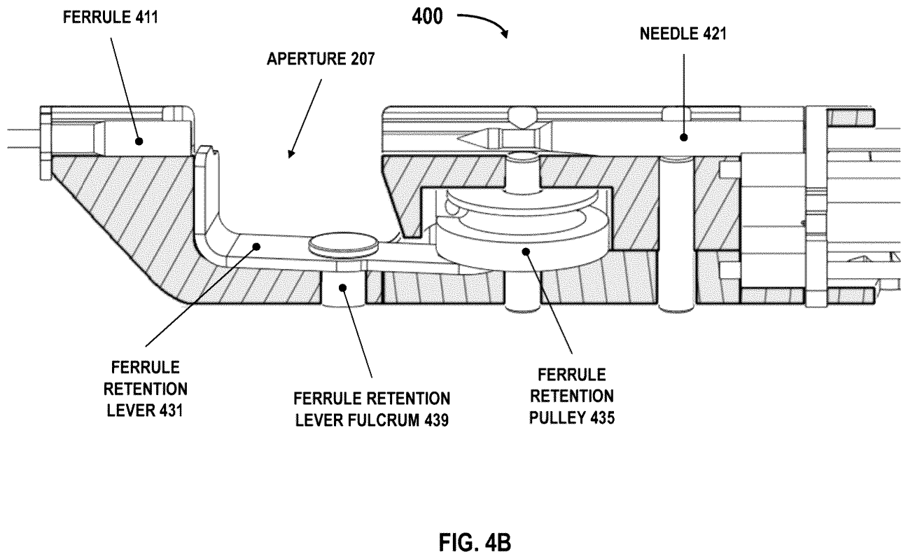

FIG. 3 and FIGS. 4A-4B show examples of a substantially rigid portion 400 of the end effector. A rigid portion of the end effector may comprise a proximal portion 420 which houses an active ferrule retention mechanism 430 and a needle 421. The rigid portion of the end effector may comprise a distal portion 410 which provides a ferrule stowage location (pocket 413) receiving a ferrule 411 and an aperture 207 between the proximal and distal portions.

As shown in the example 300, the ferrule 411 may be deposited to the pocket at the distal portion, and is secured in the pocket by a retention lever 431. As shown in the examples 310 and 320, the needle 421 may translate forward and backward engaged with the ferrule 411 or without the ferrule. The active ferrule retention mechanism 430 may beneficially allow the engagement and disengagement between the needle and ferrule performed without rotating the needle, and decoupling the motion of the needle from the ferrule engagement. For example, the ferrule may exit the pocket at any desired time independent of an operation of the needle.

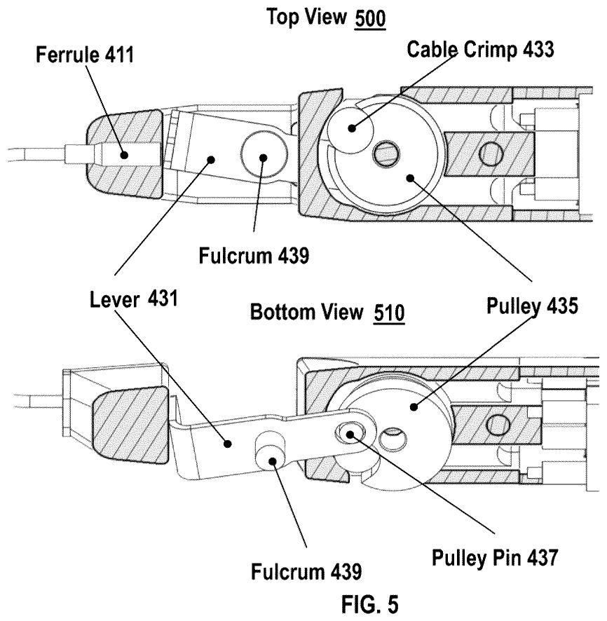

In some embodiments, the active ferrule retention mechanism may comprise substantially a pulley 435, a lever 431 and a fulcrum 439. FIG. 5 shows a top view 500 and a bottom view 510 of an active ferrule retention mechanism. As shown in the example, the ferrule retention pulley 435 may comprise a feature to couple to the pulley to a cable (e.g., cable 1021 in FIG. 10) such that a displacement of the cable results in an angular displacement of the pulley. For example, the cable 1021 may extend down the shaft of the instrument and pulled or pushed via the IDM at the proximal end such that the pulley 435 may be rotated to rotate the lever 321. The pulley 435 may comprise a pin 437 for coupling to the ferrule retention lever 431 to the pulley 435. When the pulley is controlled to rotate by certain angles, the pulley pin forces the ferrule retention lever to displace to predefined locations.

In some embodiments, the lever is constrained to move between predefined locations via a centrally located fulcrum 439. For instance, when the pulley pin 437 forces a proximal lever displacement, the fulcrum 439 converts the proximal displacement to a distal displacement in the opposite direction. In some cases, a rotation of the pulley in one direction moves the lever to a “ferrule retention” location and a rotation of the pulley in the opposite direction moves the lever to a “ferrule release” location.

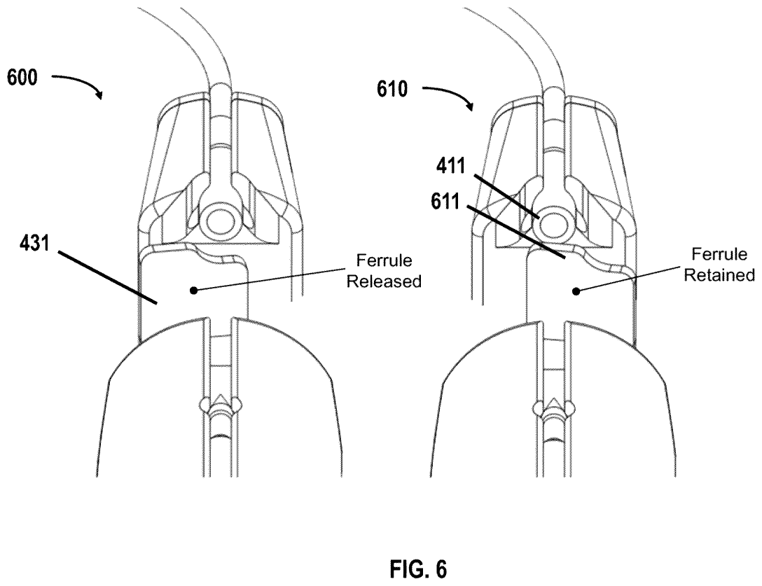

FIG. 6 shows examples of a ferrule retention lever 431 positioned at a “ferrule retention” location 610 and a ferrule release location 600. As shown in the example, the ferrule retention lever may function as a door to open or close the pocket that receives the ferrule. The ferrule retention lever may comprise an edge profile 611 with varying heights such that when the ferrule retention lever is rotated to the ferrule retention location, a higher edge of the ferrule retention lever may interfere with a proximal end of the ferrule 411 thereby securing the ferrule in the pocket (to prevent the ferrule from retracting). When the ferrule retention lever is rotated to the ferrule released location 600, a lower edge of the ferrule retention lever may permit the ferrule to exit the pocket on its own or be retracted out of the pocket by a needle. The edge profile 611 may be designed such that the higher edge may interfere with the proximal end of the ferrule while allowing a needle to pass and engage with the ferrule.

As the ferrule retention status is controlled by the active ferrule retention mechanism independent of the needle displacement motion, the needle and ferrule retention mechanisms can be independently controlled. By decoupling the control of the ferrule retention and needle operation, the suturing device has improved flexibility to operate the needle and ferrule. For example, as shown in FIG. 4A, the ferrule door (i.e., lever) can be opened at any desired time or at any state. For instance, when the ferrule door is open (level is at the ferrule released location) and the needle is retracted, the ferrule is free to exit the end effector allowing the ferrule to be dropped at the completion of the wound closure, at the event of an issue or at any desired time.

The end effector may have a reduced length in the rigid portion. The reduced length may be achieved via a flexible construction of the needle and by arranging a needle drive mechanism to a location within the bending section, near a proximal end of the bending section, at the shaft or at the interface between the bending section and the shaft.

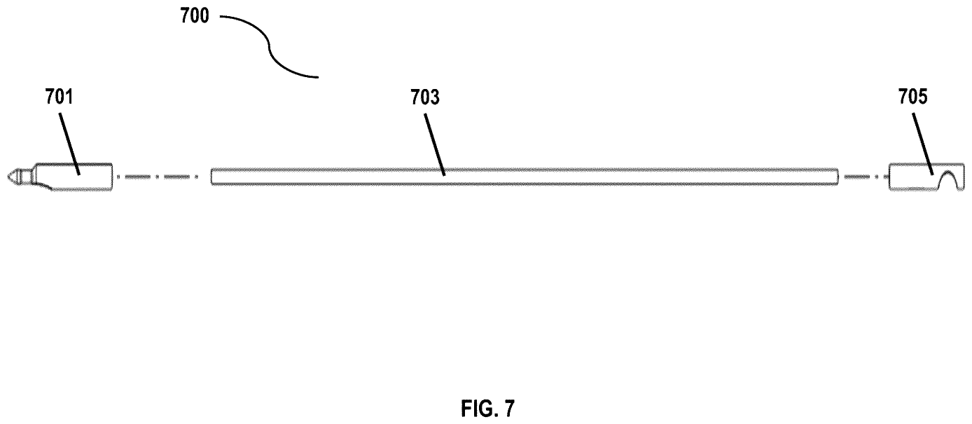

FIG. 7 shows an example of a needle 700 having a flexible construction. In some embodiments, the needle 700 may comprise a needle tip 701, a needle flexible shaft 703 and a cable coupler 705. The needle tip 701 may be a pointed tip that is substantially rigid and can be engaged with a ferrule. The cable coupler 705 may be a cable coupling tail to be coupled to a cable driving a translational motion of the needle or for controlling needle displacement. The needle flexible shaft 703 may be a flexible construction that is formed of material and geometrics to resist axial compression. In some cases, the needle flexible shaft 703 may be constructed from a nitinol wire, or other suitable constructions that combine flexibility with incompressibility.

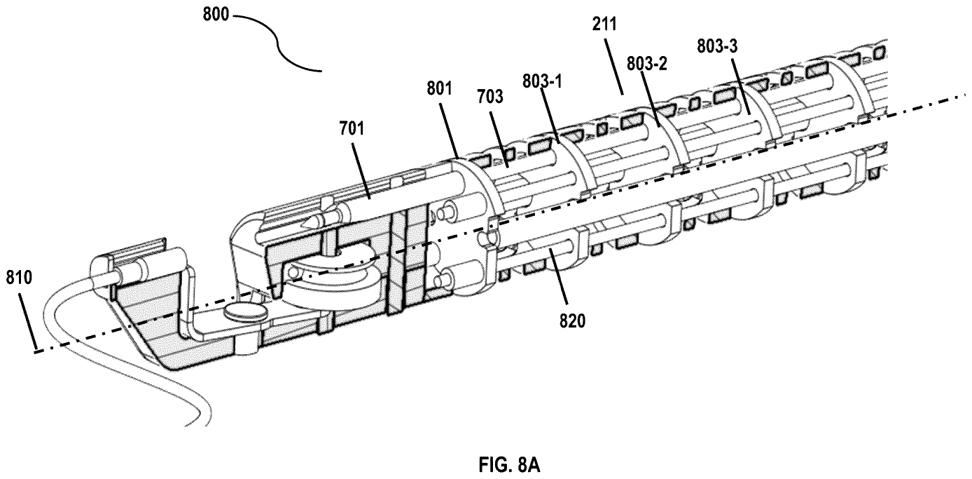

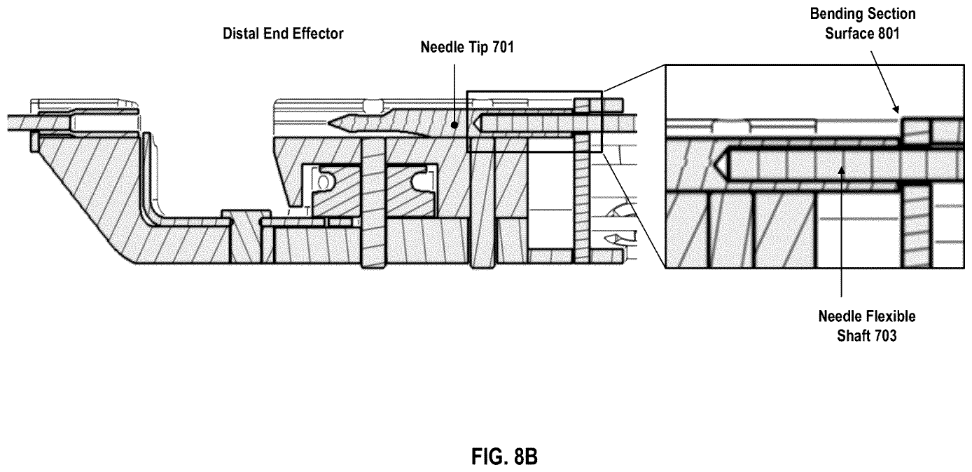

The axial incompressibility may allow drive force to be transmitted to the needle tip while the flexibility beneficially allows the needle flexible shaft extending through the bending section of the suturing instrument. In some embodiments, the needle flexible shaft may be extended into the entire length of the bending section and may be bent along with the bending section. In some cases, a length of the needle flexible shaft may be longer than an axial length of the bending section. FIGS. 8A-8B show examples of a needle at least partially located within a bending section 211.

As shown in the example 800, the needle flexible shaft 703 or the flexible nitinol shaft of the needle may translate or extend through the bending section 211. The needle flexible shaft may be located at an offset from the bending section neutral axis 810 (e.g., centerline of the bending section), the offset pathway may require the length of the path through the bending section change as the bending section is bent. The needle flexible shaft 703 may be capable of estimating the bending section orientation so as to vary the lengths changing as the bending section is bent in different directions or bending degrees.

As shown in the example, the needle flexible shaft 703 may be guided by eyelets of one or more disks 803-1, 803-2, 803-3 within the bending section to keep the needle flexible shaft at a predefined distance (i.e., offset from the centerline) from the bending section neutral axis 810.

The one or more disks 803-1, 803-2, 803-3 may comprise eyelets or holes for passing through one or more pull wires 820 to drive articulation of the bending section 211 and for passing through one or more cables for the ferrule retention mechanism. In some cases, the one or more disks may further comprise eyelets or holes for passing coil pipes to the end effector for the purpose of constraining the path length of wires routing to the ferrule retention mechanism. The one or more disks 803-1, 803-2, 803-3 may be formed of any suitable material such as metallic materials, stainless steel or nitinol, stiff polymers such as PEEK, glass or carbon filled PEEK, Ultem, Polysulfone, ceramics, composite material and other suitable materials.

FIG. 8B shows a cross-sectional view of the needle coupled to a distal end of a bending section. In the illustrated example, the needle tip 701 and the needle flexible shaft 703 may be assembled or connected and the needle tip may have a diameter no less than a diameter of the needle flexible shaft. In some cases, when the needle is at a fully retracted location, the proximal end of the needle tip may be substantially located at a bending section surface 801 interfacing the rigid portion of the end effector. As the bending section is bent, the distal end of the needle may stay coupled to the distal end of the bending section, while the proximal end of the needle flexible shaft moves due to the change in its path length through the bending section.

Due to the offset arrangement of the needle, if the needle retraction cable is attempting to maintain a tension to keep the needle retracted, the needle tip may be biased up against the distal end of the bending section (e.g., tend to bend the bending section up). In some cases, this tension in the needle retraction cable may exert force on the bending section resulting in undesired deflection of the distal tip. In some cases, the flexible shaft of the needle adapts to a path length change when the bending section is articulated. To ensure the needle tip stays coupled to the distal end of the bending section while the length of the needle flexible shaft in the bending section can dynamically change (e.g., a length of the needle flexible shaft within the entire bending section and at an offset from the centerline varies to accommodate the curvature or articulation of the bending section), a tension in the needle retraction cable may be monitored to maintain the tension within a target range. By coordinating the tension in the needle retraction cable with a bending/articulation configuration of the bending section, the undesired deflection of the distal tip may be reduced/eliminated.

In some cases, as the retraction cable is maintaining a tension, the motor controlling the needle retraction may rotate to maintain the tension. The target tension range may be maintained by providing wire slack if tension is too high, or reducing wire slack if the tension is too low. In some cases, the maintenance of the target tension may provide an estimation of the bending degree, or bending orientation of the bending section. A tension in the needle retraction cable may be monitored or measured in real-time such as utilizing a torque sensor or be based on motor current. In some cases, the tension may be maintained based on a relationship between the tension and an articulation angle. For example, the relationship between the amount of adjustment in tension or change in tension and a bend angle may be obtained from a calibration process, using a theorical method, modeling or a combination of any of the above. Alternatively, a length of the flexible shaft of the needle within the entire bending section may dynamically change to adapt to the path length change thereby maintain a constant tension in the needle retraction cable. In some cases, the amount of change in length may be determined based on a relationship between the change in length and an articulation angle, and the relationship may be obtained from a calibration process, using a theorical method, modeling or a combination of any of the above. For example, an amount of angular rotation on the motor for driving the needle retraction cable may be correlated to the bending angle (direction and degree) of the bending section. The angular rotation of the motor may be measured by an encoder or other suitable sensor that is in the IDM. In some cases, the bending section orientation or bending angle may be estimated based on the robotic control signal for articulating the bending section. In some cases, the bending section orientation or bending angle may be estimated based on motor current and angle change of the motor for driving a pull wire attached to the bending section.

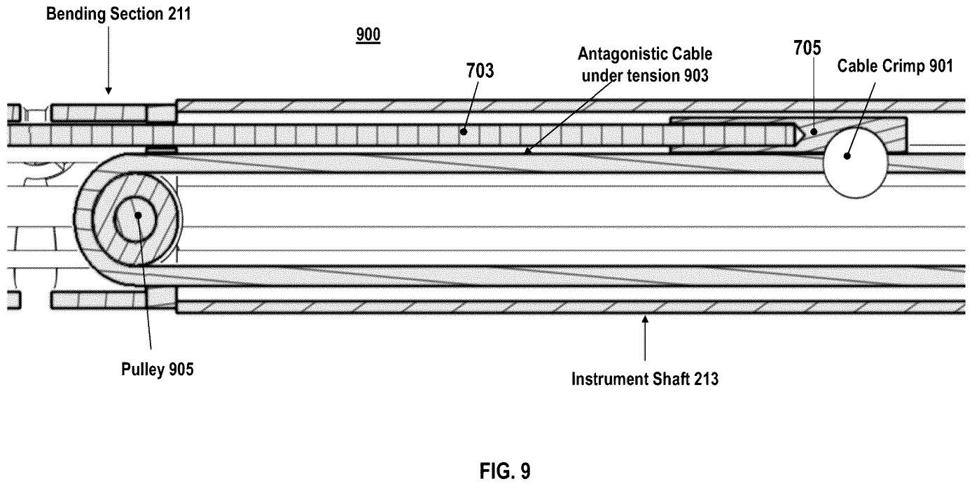

As described above, the end effector may comprise a needle drive mechanism which is located within a bending section of the suturing instrument. FIG. 9 and FIG. 10 show an example of a needle drive mechanism 900. The needle drive mechanism 900 may be positioned proximal to the bending section 211 and at the distal end of the instrument shaft 213. In some cases, the needle drive mechanism 900 may be located substantially at an interface 215 between the bending section 211 and the shaft 213. As shown in FIG. 10, the needle end effector may have a reduced effective length or reduced length of the rigid portion 1010. Example dimension of a needle end effector such as the length 1010 of the needle end effector may be no greater than 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, any number in between the above numbers or any number greater than 30 mm or small than 10 mm. By moving a portion of the needle and the needle drive mechanism to the bending section, the length 1010 of the end effector tip may be substantially dependent on the ferrule length.

The needle end effector may have a reduced outer diameter 1011. For example, the outer diameter 1011 of the instrument may be no greater than 5 millimeters (mm), 4.9 mm, 4.8 mm, 4.7 mm, 4.6 mm, 4.5 mm, 4.4 mm, 4.3 mm, 4.2 mm, 4.1 mm, 4 mm, 3.5 mm, 3 mm, any number in between the above numbers or any number greater than 5 mm or small than 3 mm. In some cases, the overall size of the needle end effector e.g., total length and diameter may be substantially dependent on the tissue aperture size 207.

The needle drive mechanism 900 may comprise a cable 903 with a crimp 901. The crimp 901 on the cable may be coupled to the needle via the cable coupler 705 such that crimp displacement results in needle translational movement. The crimp may be positioned at any suitable location of the cable. For instance, the crimp may be located at approximately mid-length on the cable.

The needle may be driven forward and backward by antagonistic cables 903. The antagonistic cables herein may refer to the cables that pulling on one end of the cable advances the needle while pulling on the other end of the cable retracts the needle. For instance, the needle may be driven forward by the needle insert cable 904 to pass through the tissue aperture (e.g., piercing the tissue) and then driven backward (e.g., drawing the suture through the resulting hole in the tissue) by the needle retraction cable 903. The antagonistic cables may beneficially allow the end effector to be suitable for being used with a flexible articulating shaft. The cable 903 may be looped over a pulley 905. The pulley may be located or supported at the interface or transition between the bending section 211 and the shaft 213 such that each end of the cable can be tensioned and the cable ends can be displaced antagonistically to change the position of the crimp relative to the shaft and bending section.

FIG. 10 shows an example 1000 of both the active ferrule retention mechanism and the needle drive mechanism in the end effector are controlled with cables. The cables for both the mechanisms may extend through the shaft of the instrument and terminated at the proximal handle. The cable pathways may comprise coil pipes to reinforce the cable path within the bending section and resolve the unintended motion of the shaft during articulation. The coil pipes beneficially prevent path elongation or compression as the shaft of the instrument is bent due to anatomy.

The end effector herein may beneficially adapt to needles and ferrules with engaging geometries. The active ferrule retention mechanism may be capable of adapting to the ferrule and needle having different engagement interfaces. FIG. 11 shows different engagement features 1110, 1120 of the needles and ferrules. FIG. 11 shows different examples of a ferrule 1111, 1121. In some embodiments, a ferrule may comprise a substantially tubular body with a suture engaging aperture 1113 and a needle engaging aperture 1117. In some cases, the suture engaging aperture 1113 may have a dimension (e.g., diameter) smaller than that of the needle engaging aperture 1117. During manufacturing, a suture (e.g., suture 201) may be inserted into the suture aperture of the ferrule and swaged in place to couple the ferrule to the suture. In some cases, the suture aperture may have an exterior shape such as a tapered surface 1115 to assist depositing the ferrule back to the end effector distal tip and to stop the ferrule from advancing further when it is deposited back to the pocket.

The ferrule engaging features 1117, 1127 and needle engaging features 1119, 1129 may have different geometries. The first example 1100 may comprise a needle with a stepped diameter tip 1118 where the distal diameter is larger than the proximal “neck” diameter 1117. For example, the local wall deformation 1117 may be crimp indentations. The local wall deformations may be integrally formed with the ferrule during manufacturing. Such deformations may allow the ferrule to couple to the needle during operation of the device and prevent decoupling under forces supplied by tissue. For example, the ferrule 1111 is manufactured such that the needle tip receptacle 1117 is in interference with the larger needle tip diameter. The needle tip dilates the receptacle upon entry and snaps to the ferrule once the larger diameter needle tip has passed the ferrule diameter reduction. Ferrule release may be conducted by pulling the needle from the ferrule needle receptacle, forcing the ferrule to dilate over the larger needle tip.

In the second example 1120, the needle and ferrule engagement features may comprise mating tapers 1127, 1129. For example, the needle may have a tapered needle tip 1129 in the range of 1°-4° where the tapered needle tip is locked to the mating tapered inner surface of the needle 1127 under the compressive attachment force provided by the needle drive mechanism. The active ferrule retention mechanism may permit a wide range of needle engagement depth 1130. The depth of needle engagement is driven by the interference between the mating tapers, rather than the location of local wall deformations. This may beneficially reduce the cost and variability in manufacturing due to the local wall deformations and stepped needle tip, while still providing for sufficient retention force between the needle and the ferrule.

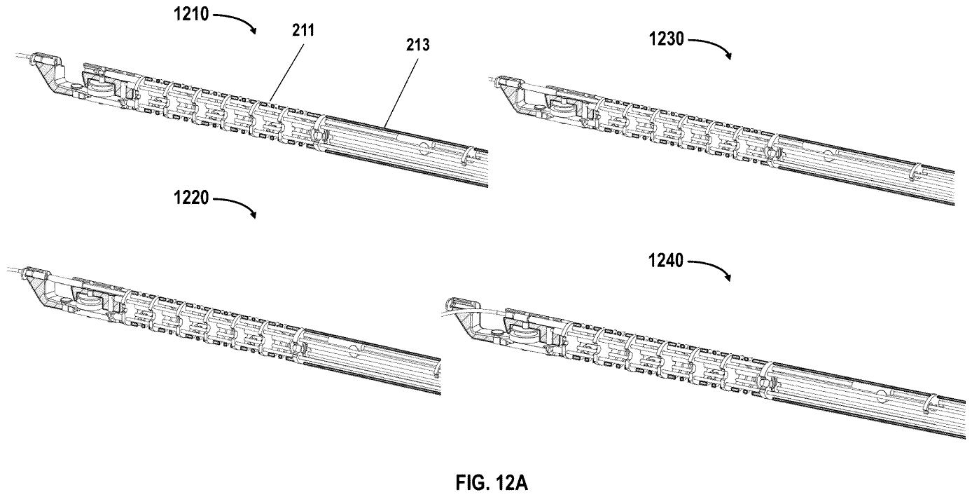

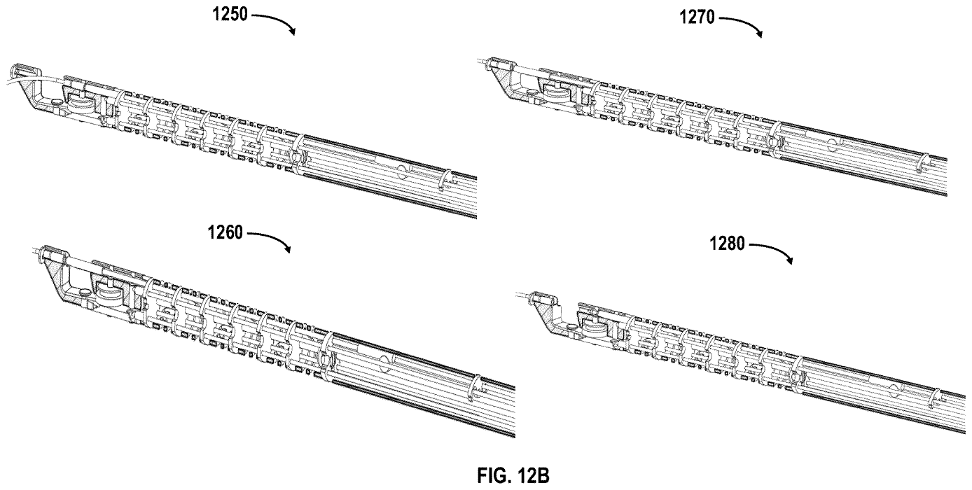

The suturing instrument may allow needle operation and ferrule retention operation to be controlled independently by employing the active ferrule retention mechanism and needle drive mechanism described herein. Such independent control beneficially allows various combinations or sequences of operations based on the use intent or application. By decoupling the needle operation and the ferrule retention operation, the system herein provides improved flexibility to perform various workflows or sequences of operations. FIGS. 12A-12B show an exemplary sequence of operations for throwing a stitch.

For example, at a first operation 1210, the ferrule door may be closed by controlling the ferrule retention lever to be positioned at the ferrule retention position as described above, and the needle may be controlled to be at a retracted position. At a second operation 1220, the ferrule door may remain closed while the needle is driven by the needle drive mechanism to advance through tissue captured in the aperture and may engage with the ferrule. Once the needle is engaged with the ferrule in the distal portion of the end effector, the ferrule door may be opened 1230 by driving the ferrule retention lever to the ferrule release position. In the next operation 1240, while the ferrule door remains open, the engaged needle and ferrule may be retracted through the tissue by the needle drive mechanism. The tissue may be removed from the aperture at the next operation 1250. The ferrule door may remain open while the tissue and ferrule may be advanced through the aperture 1260 until they reach the pocket at the distal portion of the end effector. Then the ferrule door is closed 1270 by rotating the ferrule retention lever to the ferrule retention position. While the ferrule door remains closed, the needle is retracted to strip ferrule from needle 1280. The ferrule may be decoupled from the needle by the ferrule retention lever in the distal end of the end effector, such that the ferrule is re-deposited back into the pocket in the end effector distal portion.

The controller may generate control signals to the motors for the active ferrule retention mechanism and the needle drive mechanism to coordinate the operations as described above. The operations may be automated such as by pre-programmed commands. For example, different sequences of operations may be pre-programmed and stored in a storage for execution upon request. Alternatively, the operations may be adjustable based on real-time sensor data or can be manually changed upon an input from a user.

### Flexible Endoscope

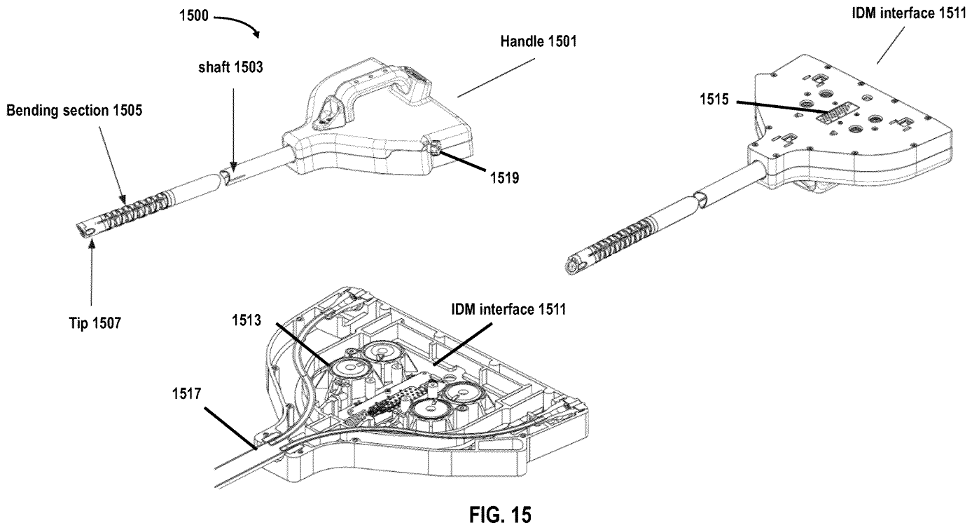

The provided suturing instrument may be utilized by any robotic endoluminal systems or platforms. In an aspect of the invention, the suturing instrument may be inserted through a working channel of a flexible endoscope to perform suturing operations as described above. The suturing instrument may be independently steerable from the endoscope. For example, the suturing instrument shaft may be advanced and retracted and rotated relative to the flexible endoscope. FIG. 15 illustrates an example of a flexible endoscope 1500, in accordance with some embodiments of the present disclosure. As shown in FIG. 15, the flexible endoscope 1500 may comprise a handle/proximal portion 1501 and a flexible elongate member to be inserted inside of a subject. The flexible elongate member may comprise at least a distal tip portion 1507, a bending section 1505 and a shaft 1503. In some cases, the endoscope 1500 may also be referred to as steerable catheter assembly as. In some cases, the endoscope 1500 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiments, the endoscope may contain varying levels of stiffness along the shaft, as to improve functional operation.

The endoscope or steerable catheter assembly 1500 may comprise a handle portion 1501 that may include one or more components configured to process image data, provide power, or establish communication with other external devices. For instance, the handle portion may include a circuitry and communication elements 1515 that enables electrical communication between the steerable catheter assembly 1500 and an instrument driving mechanism (e.g., IDM 1320 in FIG. 13), and any other external system or devices. In another example, the handle portion 1501 may comprise circuitry elements such as power sources for powering the electronics (e.g., camera, electromagnetic sensor and LED lights) of the endoscope.

The one or more components located at the handle may be optimized such that expensive and complicated components may be allocated to the robotic support system (e.g., 1300 in FIG. 13), a hand-held controller or an instrument driving mechanism (e.g., IDM 1320 in FIG. 13) thereby reducing the cost and simplifying the design of the disposable endoscope.

The handle portion or proximal portion 1501 may provide an electrical interface 1515 and mechanical interface 1513 to allow for electrical communication and mechanical communication with the instrument driving mechanism (e.g., IDM 1320 in FIG. 13). As shown in FIG. 14, the instrument driving mechanism 1320 for controlling the endoscope may comprise a set of motors 1321 that are actuated to rotationally drive a set of pull wires 1517 of the catheter. The handle portion of the catheter assembly may be mounted onto the instrument drive mechanism so that its pulley/capstans assemblies 1513 are driven by the set of motors 1321 via the output shaft 1323. The number of pulleys may vary based on the pull wire configurations. In some cases, one, two, three, four, or more pull wires may be utilized for articulating the flexible endoscope or catheter.

The handle portion may be designed allowing the robotic endoscope to be disposable at reduced cost. For instance, classic manual and robotic endoscopes may have a cable in the proximal end of the endoscope handle. The cable may comprise, for example, illumination fibers, and camera video cable. In some embodiments, the distal tip portion 1507 may be embedded with a positional sensor such as electromagnetic (EM) sensors thus the cable may comprise other sensors fibers or cables, EM sensor cable or shape sensing fibers. Alternatively, the distal tip portion 1507 may not be embedded with EM sensor. Such complex cable can be expensive adding to the cost of the endoscope. The provided robotic endoscope may have an optimized design such that simplified structures and components can be employed while preserving the mechanical and electrical functionalities. In some cases, the handle portion of the robotic endoscope may employ a cable-free design while providing a mechanical/electrical interface to the catheter.

The electrical interface (e.g., printed circuit board) 1515 may allow image/video data and/or sensor data to be received by the communication module of the instrument driving mechanism 1320 and may be transmitted to other external devices/systems. In some cases, the electrical interface may establish electrical communication without cables or wires. For example, the interface may comprise pins soldered onto an electronics board such as a printed circuit board (PCB) 1515. For instance, a receptacle connector (e.g., the female connector) 1325 is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

In some cases, the handle portion 1501 may comprise one or more mechanical control modules such as luer 1519 for interfacing the irrigation system/aspiration system. In some cases, the handle portion may include a lever/knob for articulation control. Alternatively, the articulation control may be located at a separate controller attached to the handle portion via the instrument driving mechanism.

The endoscope may be attached to a robotic support system 1300 via the instrument driving mechanism 1320 as shown in FIG. 13. Alternatively, the endoscope may be attached to a hand-held controller via an IDM on the hand-held controller. The instrument driving mechanism may be provided by any suitable controller device (e.g., hand-held controller) that may or may not include a robotic system. The instrument driving mechanism may provide mechanical and electrical interface to the steerable catheter assembly 1500. The mechanical interface may allow the steerable catheter assembly 1500 to be releasably coupled to the instrument driving mechanism. For instance, the handle portion of the steerable catheter assembly can be attached to the instrument driving mechanism via quick install/release means, such as magnets, spring-loaded levers and the like. In some cases, the steerable catheter assembly may be coupled to or released from the instrument driving mechanism manually without using a tool.

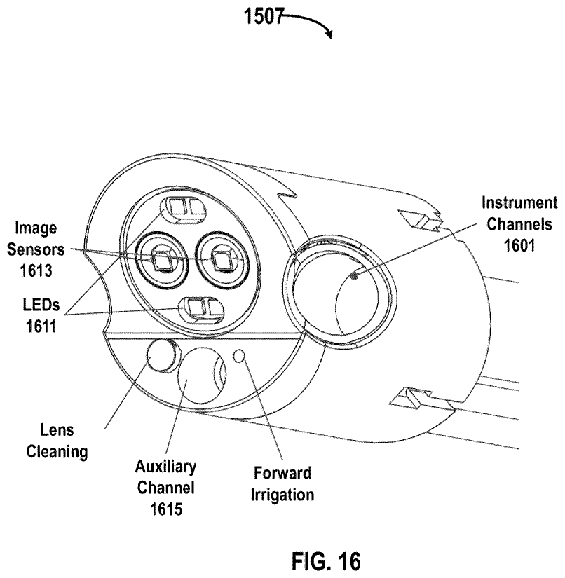

In the illustrated example, the distal tip 1507 of the catheter or endoscope shaft is configured to be articulated/bent in two or more degrees of freedom to provide a desired camera view or control the direction of the endoscope. As illustrated in FIG. 16, imaging device 1613 (e.g., camera), and/or illumination electronics (e.g., LED light source 1611) may be embedded in the distal tip of the catheter or endoscope shaft 1507. For example, line of sight of the camera may be controlled by controlling the articulation of the active bending section 1505. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with the aid of an optical component.

The distal tip 1507 may be a rigid component that allows for imaging devices (e.g., camera) and other electronic components 1611 (e.g., LED light source) being embedded at the distal tip. Depending on the type of the endoscope, the distal tip may comprise other sensors such as electromagnetic (EM) sensors or inertial measurement units embedded in the distal tip.

The robotic endoscope may or may not have real-time EM tracking capability. In some embodiments, the robotic endoscope may not include positional sensors (e.g., electromagnetic sensor). A real-time camera view may be displayed on the screen to provide visual feedback to an operator. For instance, during colonoscopy, the endoscope may traverse a single path forward or backward without requiring tracking an accurate position of the endoscope tip with respect to a model of path. Alternatively, when the endoscope is traversing a network of passageways (e.g., bronchoscopy), real-time position of the endoscope tip may be tracked for navigating the endoscope across the network. In the case that the robotic endoscope is embedded with EM sensor, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient during a procedure to locate the EM sensor position in 3D space or may locate the EM sensor position and orientation in 5D or 6D space. This may provide a visual guide to an operator when driving the endoscope towards the target site.

The endoscope may have a unique design in the elongate member. In some cases, the active bending section 1505 and the shaft 1503 of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

As described above, the active bending section 1505 may be designed to allow for bending in two or more degrees of freedom (e.g., articulation). A greater bending degree such as 180 and 270 degrees (or other articulation parameters for clinical indications) can be achieved by the unique structure of the active bending section. In some cases, the active bending section may be fabricated separately as a modular component and assembled to the shaft. In some cases, the cut patterns of the active bending section may be variable such that at least a minimum bend radius of the bending section may vary along the length. In some cases, a variable minimum bend radius along the axial axis of the elongate member may be provided.

The articulation of the endoscope may be controlled by applying force to the distal end of the endoscope via one or multiple pull wires 1517. The one or more pull wires may be attached to the distal end of the endoscope (e.g., at the distal end of the bending section 1505 or the proximal end of the tip 1507). In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley 1513 may interact with an output shaft 1323 from the robotic system.

In some embodiments, the proximal end or portion of one or more pull wires 1517 may be operatively coupled to various mechanisms (e.g., gears, pulleys, capstans, etc.) 1513 in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals.

In some embodiments, the provided robotic endoscope can be a single-use endoscope that may beneficially reduce cross-contamination between patients and infections. In some cases, the robotic gastroscope may be delivered to the medical practitioner in a pre-sterilized package and are intended to be disposed of after a single use. The proximal design may improve the reliability of the device without introducing extra cost allowing for a low-cost single-use endoscope. Alternatively, the robotic endoscope may be reusable.

As shown in FIG. 15, in some cases, a robotic endoscope (e.g., gastroscope or colonoscope) 1500 may comprise a handle portion 1501 and a flexible elongate member. In some embodiments, the flexible elongate member may comprise a shaft, steerable tip, a steerable/active bending section and optionally an anti-prolapse passive section. The robotic gastroscope may be a single-use robotic endoscope. In some cases, only the catheter may be disposable. In some cases, at least a portion of the catheter may be disposable. In some cases, the entire robotic gastroscope may be released from the instrument driving mechanism and can be disposed of. In some cases, the gastroscope may contain varying levels of stiffness along its shaft, as to improve functional operation. In some cases, a minimum bend radius along the shaft may vary so that the kink resistance or anti-prolapse capability may be configurable along the length.

The robotic gastroscope can be releasably coupled to an instrument driving mechanism 1320. The instrument driving mechanism 1320 may be mounted to the arm of the robotic support system 1300 or to any actuated support system as. The instrument driving mechanism may provide mechanical and electrical interface to the robotic gastroscope 1500. The mechanical interface may allow the robotic gastroscope 1500 to be releasably coupled to the instrument driving mechanism. For instance, the handle portion of the robotic gastroscope can be attached to the instrument driving mechanism via quick install/release means, such as magnets and spring-loaded levels. In some cases, the robotic gastroscope may be coupled or released from the instrument driving mechanism manually without using a tool.

The handle portion 1501 may be designed allowing the robotic gastroscope to be disposable at reduced cost. For instance, classic manual and robotic gastroscopes may have a cable in the proximal end of the gastroscope handle. The cable often includes illumination fibers, camera video cable, and other optional sensor fibers or cables such as electromagnetic (EM) sensors, or shape sensing fibers. Such complex cable can be expensive, adding to the cost of the gastroscope. The provided robotic gastroscope may have an optimized design such that simplified structures and components can be employed while preserving the mechanical and electrical functionalities. In some cases, the handle portion of the robotic gastroscope may employ a cable-free design while providing a mechanical/electrical interface to the catheter.

The endoscope may comprise a tip portion, bending section, and insertion shaft. In some embodiments, the endoscope may have variable bending stiffness along the longitudinal axis direction. For instance, the endoscope may comprise multiple sections having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some embodiments, the endoscope may have variable minimum bend radius along the longitudinal axis direction. The selection of different minimum bend radius at different locations along the endoscope may beneficially provide anti-prolapse capability while still allowing the endoscope to reach hard-to-reach regions. In some cases, a proximal end of the endoscope needs not be bent to a high degree thus the proximal portion of the endoscope may be reinforced with additional mechanical structure (e.g., additional layers of materials) to achieve a greater bending stiffness. Such a design may provide support and stability to the endoscope. In some cases, the variable bending stiffness may be achieved by using different materials during extrusion of the endoscope. This may advantageously allow for different stiffness levels along the shaft of the endoscope in an extrusion manufacturing process without additional fastening or assembling of different materials.

The endoscope may have a dimension so that one or more electronic components can be integrated to the endoscope. For example, as shown in FIG. 16, the outer diameter of the distal tip 1507 may range from 3 mm to 25 mm, and the diameter of the instrument channels 1601 may range from 2 mm to 6 mm such that one or more instruments can be removably inserted through the endoscope to the surgical site. However, it should be noted that based on different applications, the outer diameter can be in any range smaller than 3 mm or greater than 25 mm, and the diameter of the instrument channels 1601 can be in any range such as about 4 mm or 5 mm to allow the suturing instrument herein passing through. The space not occupied by fluidics or instrument pass throughs can be used to embed electronic components into the wall of the endoscope.

The one or more electronic components may comprise an imaging device, illumination device or other optional sensors. In some embodiments, the imaging device may be a video camera 1613. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 1611 positioned at the distal tip. The light source may be a light-emitting diode (LED), an organic LED (OLED), a quantum dot, or any other suitable light source. In some cases, the light source may be a miniaturized LED for a compact design or Dual Tone Flash LED Lighting.

The imaging device and the illumination device may be integrated to the endoscope. For example, the distal portion of the endoscope may comprise suitable structures matching at least a dimension of the imaging device and the illumination device. The imaging device and the illumination device may be embedded into the catheter. A camera may be located at the distal portion 1507. The distal tip may have a structure to receive the camera, and illumination device. For example, the camera may be embedded into a cavity at the distal tip of the catheter. The cavity may be integrally formed with the distal portion of the cavity and may have a dimension matching a length/width of the camera such that the camera may not move relative to the endoscope. The camera may be adjacent to one or more instrument channels 1601 of the endoscope to provide near field view of the tissue or the organs. In some cases, the attitude or orientation of the imaging device may be controlled by controlling a rotational movement (e.g., roll) of the endoscope.

The power to the camera may be provided by a wired cable. In some cases, the cable wire may be in a wire bundle providing power to the camera as well as illumination elements or other circuitry at the distal tip of the endoscope. The camera and/or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure having a dimension matching a dimension of the miniaturized LED light source. As shown in the illustrated example, two cavities may be integrally formed with the endoscope to receive two LED light sources 1611. For instance, the outer diameter of the distal tip may range from 3 mm to 25 mm and diameter of the working channel of the endoscope may be around 4.5 or 6 mm such that two LED light sources may be embedded at the distal end. The outer diameter can be in any range smaller than 3 mm or greater than 25 mm, and the diameter of the instrument channels 1601 can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiments, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover may be placed at the forwarding end face of the distal tip providing precise positioning of the LEDs as well as sufficient room for the glue. The cover may be composed of transparent material matching the refractive index of the glue so that the illumination light may not be obstructed.

The working channel (e.g., instrument channel 1601, auxiliary channel 1615) may be designed to provide protection for the internal components such as flexible instruments (e.g., suturing instrument, forceps, etc.). When flexible instruments pass through a conventional working channel, they may be obstructed by the working channel due to kinking, ovalizing and/or high friction force. The working channel may provide a high hoop strength and a capability of achieving low bend radius. The working channel may also be designed to provide low friction in the inner surface. The suturing instrument as described herein may be passed through the working channel and advanced over the distal tip of the endoscope or retracted back into the working channel.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A suturing instrument comprising:
an elongate member comprising an articulatable bending section and a shaft;
an end effector comprising a rigid distal portion located at a distal end of the bending section, and a flexible shaft of a needle extended along an entire length of the bending section; and
a handle portion releasably attached to an instrument driving mechanism that is supported by a robotic arm, wherein the robotic arm is configured to further support another instrument driving mechanism that is releasably attached to a handle portion of an endoscope and wherein the suturing instrument is inserted through a working channel of the endoscope.

2. The suturing instrument of claim 1, wherein the rigid distal portion comprises a ferrule retention mechanism configured to strip a ferrule from the needle.

3. The suturing instrument of claim 2, wherein the ferrule retention mechanism comprises a toggle to actuate a lever into a ferrule release position and a ferrule retention position.

4. The suturing instrument of claim 3, wherein the ferrule retention mechanism is independently controlled relative to a needle driving mechanism.

5. The suturing instrument of claim 1, wherein a needle driving mechanism is located approximately at a distal end of the shaft or at an interface between a proximal end of the articulatable bending section and the distal end of the shaft.

6. The suturing instrument of claim 5, wherein the needle driving mechanism is connected to the flexible shaft of the needle.

7. The suturing instrument of claim 6, wherein the flexible shaft of the needle is located at an offset from a neutral axis of the bending section.

8. The suturing instrument of claim 7, wherein the flexible shaft of the needle adapts to a path length change when the bending section is articulated.

9. The suturing instrument of claim 8, wherein a tension in a cable for retracting the needle is maintained by adapting to the path length change.

10. A suturing instrument comprising:
a flexible elongate member comprising an articulatable bending section connected to a distal end of a shaft; and
an end effector comprising a rigid distal portion located at a distal end of the bending section, and a needle driving mechanism for driving a needle, wherein the needle driving mechanism is located at the distal end of the shaft or at an interface between a proximal end of the articulatable bending section and the distal end of the shaft, wherein the rigid distal portion comprises a ferrule retention mechanism configured to strip a ferrule from the needle, wherein the ferrule retention mechanism comprises a toggle to actuate a lever into a ferrule release position and a ferrule retention position and is independently controlled relative to the needle driving mechanism.

11. The suturing instrument of claim 10, wherein the needle driving mechanism is connected to a flexible shaft of the needle.

12. The suturing instrument of claim 11, wherein the flexible shaft of the needle is extended along an entire length of the bending section.

13. The suturing instrument of claim 12, wherein the flexible shaft of the needle is at an offset from a neutral axis of the bending section.

14. The suturing instrument of claim 13, wherein the flexible shaft of the needle adapts to a path length change when the bending section is articulated.

15. The suturing instrument of claim 14, wherein a tension in a cable for retracting the needle is maintained by adapting to the path length change.

16. The suturing instrument of claim 10, further comprising a handle portion releasably attached to an instrument driving mechanism.

17. The suturing instrument of claim 16, wherein the instrument driving mechanism is supported by a robotic arm.

18. The suturing instrument of claim 10, wherein the robotic arm is configured to further support another instrument driving mechanism that is releasably attached to a handle portion of an endoscope.

19. The suturing instrument of claim 18, wherein the suturing instrument is inserted through a working channel of the endoscope.
